(19) United States
(12) Patent Application Publication
(10) Pub. No.: US 2006/0160756 A1
(43) Pub. Date: Jul. 20, 2006

(54) Treatment and prevention of multi-drug resistance

(76) Inventors: Werner Krause, Berlin (DE); Joachim Friedrich Kapp, Berlin (DE); Reinhard Wilhelm Von Roemeling, Ridgefield, CT (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/252,245

(22) Filed: Oct. 18, 2005

Related U.S. Application Data
(60) Provisional application No. 60/619,683, filed on Oct. 19, 2004.

(51) Int. Cl. A61K 31/704

(52) U.S. Cl. 514/34

(57) ABSTRACT

A method of preventing formation of multi-drug resistance (MDR) or of treating a patient that has developed or is subject to the development of MDR comprises administering a regimen comprising one or more active drugs together with one or more MDR inhibitors such that the pharmacokinetic (PK) profile of the MDR inhibitor(s) is/are matched to the PK profile of the active drug(s).

This application claims the benefit of the filing date of U.S. Provisional Application Ser. No. 60/619,683 filed Oct. 19, 2004, which is incorporated by reference herein.

### BACKGROUND

Multi-drug resistance is a phenomenon that is observed in a variety of diseases. Examples are the treatment of different types of bacteria or viruses and of cancer. For simplicity reasons, cancer drugs will be dealt with in the following paragraphs. However, the invention is not limited to this type of disease.

MDR, which shows cross resistance to major anticancer drugs, regardless of possessing different mechanisms of action, including anthracyclines (e.g., adriamycin), vinca alkaloids (e.g., vincristine), podophyllotoxins (e.g., etoposide) and taxanes, is one of the significant obstacles in present cancer chemotherapy. Such a resistance can be observed in cancer cells after repeated chemotherapy (acquired resistance) such as acute myeloid leukemia, ovarian cancer, and breast cancer, or in cancer cells which may have already been resistant before initiation of chemotherapy (intrinsic resistance) such as non-small cell lung cancer, pancreatic cancer, and colon cancer.

It has been found that drug efflux pumps, expressed on cancer cell membranes, are closely involved in the MDR phenomenon. The drug efflux pumps are membrane glycoproteins that actively pump a wide range of anticancer drugs as substrates out of the cells. Well characterized examples are P-glycoprotein (P-gp; Roninson, I. B. et al.: Nature 309, 626, 1984) and multi-drug resistance associated protein (MRP; Cole S. P. C. et al.: Science, 258, 1650, 1992). Expression of P-gp has been reported to be correlated with MDR in patients with acute myelogenous leukemia (Campos L. et al.: Blood 79, 473, 1992), acute lymphatic leukemia and non-Hodgkin's lymphoma (Goldstein L J. et al.: J. Natl. Cancer Inst., 81, 116, 1989) and breast cancer (Trock B J. et al.: J. Natl. Cancer Inst. 89, 917, 1997). A meta-analysis comprised of 31 clinical studies (Pirker R. et al.: J. Natl. Cancer Inst. 83, 708, 1991) indicates a correlation between resistance to chemotherapy and the expression of P-gp, which was expressed in 41% of tested breast cancer specimens. MRP is also reported to be expressed in most of breast cancer (Dexter D. W. et al.: Clin. Cancer Res. 4, 1533, 1998; Lacave R. et al.: Br. J. Cancer 77, 694, 1998; Filipits M. et al.: Clin. Cancer Res. 2, 1231, 1996), as well as in non-small cell lung cancer and small cell lung cancer (Nooter K. et al.: Clin. Cancer Res. 1, 1301, 1995). These findings substantiate the idea of reversing MDR by increasing intracellular concentration of anticancer drugs by inhibiting P-gp and/or MRP functions.

Since the discovery of verapamil (VPM) as an agent for overcoming MDR, various compounds including other calcium antagonists, calmodulin inhibitors, quinidine, tamoxifen and Cyclosporin A (CSA), have been reported to overcome MDR. For instance, MDR reversal was reported in a clinical study using CSA in patients with acute non-lymphatic leukemia (Sonneveld P. et al.: Br. J. Haematol. 75, 208, 1990), in a study using VPM in patients with non-Hodgkin's lymphoma (Miller T. P. et al.: J. Clin. Oncol. 9, 17, 1991), and in a study using VPM in patients with multiple myeloma and non-Hodgkin's lymphoma (Dalton W. S. et al.: J. Clin. Oncol. 7, 415, 1989). However, it was often difficult in these studies (Ozols R. F. et al.: J. Clin. Oncol. 5, 641, 1987) to administer a sufficient amount of drugs to overcome MDR because of adverse reactions resulting from their primary pharmacological activities and the clinical development of these drugs was not successful. Furthermore, it has been revealed that there are pharmacokinetic interactions between MDR modulators and anticancer drugs, which are thought to be partly attributable to the inhibition of physiological function of P-gp by MDR modulators in normal organs such as liver and kidney. In addition, it has been postulated that there is considerable overlap in the drugs which interact with P-gp and CYP3A4, which raises a possibility that such drug interaction could be caused by the inhibition of CYP3A4 by MDR modulators. From these observations it can be concluded that there is still a significant medical need to overcome multi-drug resistance and that the medical community is presently far from having achieved this goal.

### SUMMARY OF THE INVENTION

In this invention, adjusting the pharmacokinetic profile of the MDR inhibitor to the PK profile of the chemotherapeutic drug significantly contributes to its efficacy. In addition, administering the MDR inhibitor early on in the treatment, i.e. before (acquired) MDR has developed, is able to prevent or ameliorate MDR.

The present invention relates to the treatment of patients that have developed or are subject to development of multi-drug resistance (MDR) using a combination of one or more drugs that are active in that disease and one or more MDR inhibiting drugs such that the pharmacokinetics (PK) of the MDR inhibitor(s) is (are) adjusted to match those of the active drugs, e.g., to make the plasma levels of the active drug(s) and of the MDR inhibitor(s) as parallel as possible, i.e., to match the plasma level versus time curve shapes of the MDR inhibitor to that of the drug. Alternatively, especially when such matching is not readily achievable, the plasma level of the MDR can at least be maintained above its activity threshold concentration as long as the active drug(s) are above their respective activity threshold concentration(s). In one embodiment, the active drug(s) and the MDR inhibitor(s) are administered together right after diagnosis of the disease in order to prevent formation of acquired MDR or reverse intrinsic MDR.

This invention relates to a method of treating patients with a variety of diseases, including those mentioned above and below, that are subject to the development of multi-drug resistance with a combination of one or more drugs active in the disease plus one or more multi-drug resistance inhibitors such that the pharmacokinetics of the MDR inhibitor(s) are adjusted or matched to the PK of the active drug(s) in such a way that the plasma level curves of the active drug(s) and of the MDR inhibitor(s) are as parallel as possible. It is, however, not intended to make the plasma levels identical, e.g., because of the differing dosing levels often involved. The time courses should be as parallel as possible with the proviso that the levels of the MDR inhibitor(s) are over the threshold concentration of activity as long as the active drug(s) are over their respective threshold activity concentrations.

Inhibitors are currently used after the development of MDR in order to reverse it. In an embodiment of the invention, the MDR inhibitor treatment is instead initiated at the earliest possible time point in the disease regimen, preferably immediately after diagnosis, without prior treatment with any other drug, in order to prevent or ameliorate acquisition of MDR.

Thus, according to the invention, patients with a variety of diseases are treated with one or more drugs active in the disease plus one or more MDR inhibitors that are selected and/or dosed such that their pharmacokinetic profile will result in plasma levels parallel to those of the active drugs. For instance, dosing regimens can be selected that result in similar, parallel plasma level profiles. As a further alternative, dosing regimens are selected such that the concentration of the MDR inhibitor(s) are above their activity threshold as long as the active drug(s) are above their activity thresholds.

In another embodiment of the invention, these MDR inhibitors will be added to the active drug regimen as first-line treatment in order to prevent or ameliorate acquisition of MDR.

For simplicity reasons, the invention is described using examples from the field of oncology. However, the invention is not limited to this area. The term “active drug(s)” used in the previous and following paragraphs refers to drugs that show activity in the treatment of the respective disease, be it cancer or antibacterial treatment or any other disease subject to MDR. On the other hand, the term “MDR inhibitors” refers to drugs that do not show activity in the treatment of these diseases per se but, instead, are administered in conjunction with “active drugs” in order to prevent or reverse MDR.

MDR inhibitors useful in the invention include all available, e.g., those mentioned herein. Other examples include MC-207,110 (Phe-Arg-β-naphthylamide), 5′-methoxyhydnocarpin, INF 240, INF 271, INF 277, INF 392, INF 55, Reserpine, GG918, Diterpene from Lycopus europaeus, Epigallocatechin-3-O-gallate, Progesterone, verapamil, trifluoperazine, biricodar (VX-710 ), XR9576, Tariquidar (XR9576), Ceramide, Protein Kinase C Inhibitor (H7), N-Methylwelwitindolinone C isothiocyanate (welwistatin), cyclosporin A, erythromycin, quinine, fluphenazine, tamoxifen, Cremophor EL, dexverapamil, dexniguldipine, dexniguldipine, valspodar, tariquidar, biricodar, zosuquidar, laniquidar, elacridar, GF120918, Novobiocin, Fumitremorgin C, BIB-E, Flavopiridol, CI1033, Iressa, VX-853, diethylstilbestrol, estrone, antiestrogens, TAG-11, TAG-139, Toremifene, ONT-093, R-101933, mitotane, OC-144-093, LY-335979, Annamycin, XR-9576, R-101933, dofediquar (MS-209). With regard to P-gp inhibition, also see Thomas et al., Cancer Control, Volume 10, No. 2; 159-165; March/April 2003; with regard to breast cancer resistance proteing (BCRP, also known as ABCG2), see Doyle et al, Oncogene 22; 7340-7358; 2003.

If for example a drug used for the treatment of cancer patients is administered intravenously and has a terminal half-life of 6 hours, according to this invention the use of the MDR inhibitor that is given together with the active drug is adjusted to match the pharmacokinetics of the active drug. In this case, the MDR inhibitor is also injected IV in order to rapidly achieve maximum plasma levels. Additionally, the MDR inhibitor(s) is/are selected from all those available such that its terminal half-life is similar to that of the active drug. The dose of the MDR inhibitor is optimally such that the plasma levels of the inhibitor are above the threshold of activity for the same time period as the plasma levels of the active drug(s) are above its/their threshold of activity.

If an active drug is administered by a certain route, e.g., intravenously, the inhibitor might be given via another route, e.g., orally. In that case, the plasma level-time course of the inhibitor will also match the shape of the time course of the drug. This can be achieved, e.g., by giving the inhibitor prior to the drug so that the peak of its plasma level (C max) coincides with the time point of injecting the drug. If the half-lives of elimination of the drug and the inhibitor are different, e.g., the drug has a long half-life and the inhibitor a short one, then multiple doses of the inhibitor should be given in order again to match the plasma level-time course of the drug. Dosing regimens (dose levels, timing and number of doses, routes, etc.) can be varied and controlled as desired to match the active drug plasma profile by adjustment of conventional parameters such as formulations, release type (controlled, slow, sustained, pulsed, etc.), e.g., aided by control tests as usual and, e.g., conventionally evaluated by pharmacokinetic simulation programs, which help in selecting the appropriate scheme. See, e.g., D'Argenio DZ, Comput Programs Biomed. 1979 Mar;9(2):115-34, Sharyn D. Baker, Michelle A. Rudek, Pharmacokinetic Modeling: Handbook of Anticancer Pharmacokinetics and Pharmacodynamics in Cancer Drug Discovery and Development, Humana Press, March 2004, pps. 129-138.

For example, if the active drug is administered orally and achieves maximum plasma levels in the blood after 3 hours and has a terminal half-life of 8 hours, an MDR inhibitor could be selected that can be given orally. Moreover, its pharmaceutical formulation is selected such as to make its PK parallel to that of the active drug, i.e., to achieve maximum plasma levels at approximately 3 hours and a terminal half-life of 8 hours. This can be accomplished by either selecting an MDR inhibitor with intrinsic PK parameters matching those of the active drug or —, e.g., if the half-life of the MDR inhibitor is much shorter—by preparing a slow-release formulation with the desired PK profile.

Another possibility of matching plasma levels is the use of multiple administrations of the MDR inhibitor or single doses which pulse the inhibitor, etc., in order to match the PK profile of the active drug or—at least—to maintain plasma levels of the MDR inhibitor above its threshold of activity during approximately the same time period during which the active drug is above its respective threshold concentration. This is particularly useful in those cases where MDR inhibitors with appropriate PK profiles are not available or if a slow-release formulation is not feasible.

Another possibility is to administer more than one MDR inhibitor whereby the two or more different inhibitors contribute to different portions of the overall plasma level profile of the MDR inhibitors with the purpose that the sum of the individual MDR inhibitor profiles matches the time course of the active drug per this invention. As noted, it is not the absolute concentrations of the MDR inhibitor(s) profile that are relevant but the relative shape of the plasma-level time courses with the proviso that the plasma levels of MDR components are above the threshold concentration as long as the active drug is above its threshold concentration.

Many diseases, and in particular many cancers, are treated with cocktails of active drugs. Normally, these anti-cancer drugs do not have identical PK profiles, i.e. they might exhibit highly variable terminal half-lives and plasma level profiles. In such cases the PK profiles of the active drugs and of the MDR inhibitors are matched. In one approach, the active drug is chosen with the longest terminal half-life and the half-life (half-lives) of the MDR inhibitor(s) are adjusted accordingly. If there are multiple Cmax peaks, the same will be true of the MDR inhibitors at the same time points, to the extent possible. In any case, the regimen for the MDR inhibitor(s) is adjusted to the regimen of the active drug(s). This means that for active drugs that are given repetitively, the regimen of the MDR inhibitor(s) have to be adjusted accordingly. At all times that at least one drug level is above threshold, the MDR inhibitor(s) level will also be above threshold.

As can be seen, matching of the plasma profiles per this invention refers to making the curve shapes of the profiles as similar as possible in a relative manner (not as to absolute level values), e.g., matching as closely as possible as many of the relevant profile parameters as possible, including Tmax (time to Cmax), terminal half-life, normalized ascending slope, normalized descending slope, relative hourly concentrations, etc.) Generally, these values will be matched within ±20% or better if possible, e.g., ±10%, ±5% etc. However, lesser matches are within the scope of this invention.

### BRIEF DESCRIPTION OF DRAWINGS

FIG. 1 shows the disappearance of dofediquar's enhancing effect on ADM cytotoxicity.

FIGS. 2 and 3 show plasma levels of dofediquar upon administration of various dosage levels.

Without further elaboration, it is believed that one skilled in the art can, using the preceding description, utilize the present invention to its fullest extent. The preceding preferred specific embodiments are, therefore, to be construed as merely illustrative, and not limitative of the remander of the disclosure in any way whatsoever.

The entire disclosure of the applications, patents and publications, cited herein are incorporated by reference herein.

### EXAMPLES

### Example 1

Effects of pretreatment and consecutive additional treatment with dofediquar in MDR cells (K562/adriamycin-resistant) are assessed by MTT (3-(4,5-dimethyl-2-thiazolyl) 2,5-diphenyl-2H tetrazolium bromide) assay.

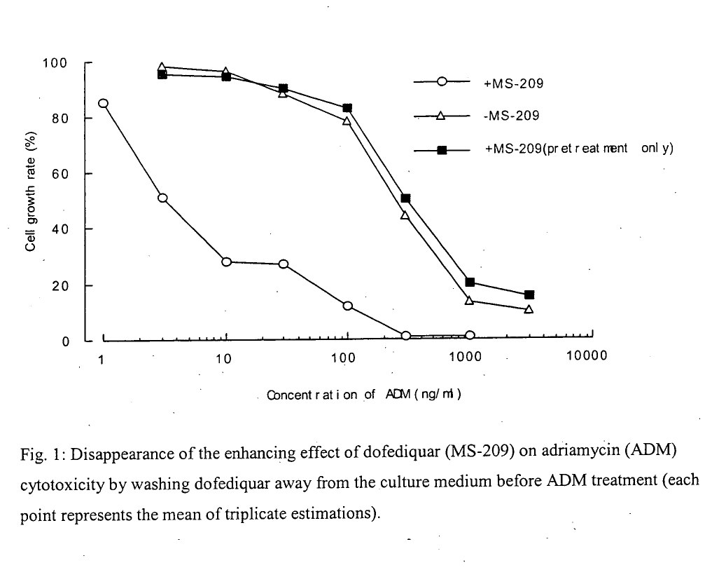

K562/adriamycin-resistant cells are pretreated with 3 μM of dofediquar for 24 hours, then, after dofediquar is washed away, the cells are incubated with ADM alone for further 72 hours. Pretreatment with dofediquar only before exposure to ADM does not show an enhancement of ADM cytotoxicity in K562/adriamycin-resistant cells (FIG. 1).

In contrast, when the cells are first co-treated with both adriamycin (100-300 ng/ml) and dofediquar (3 μM), and then followed by a 2nd incubation (24 hr) with dofediquar alone, the cell growth is slightly more inhibited than that without dofediquar during the 2nd incubation (Tab. 1).

From the above results, to get the best MDR-reversing effect, adequate concentration of dofediquar is maintained all through the exposure of tumor cells to antitumor agents.

### Example 2

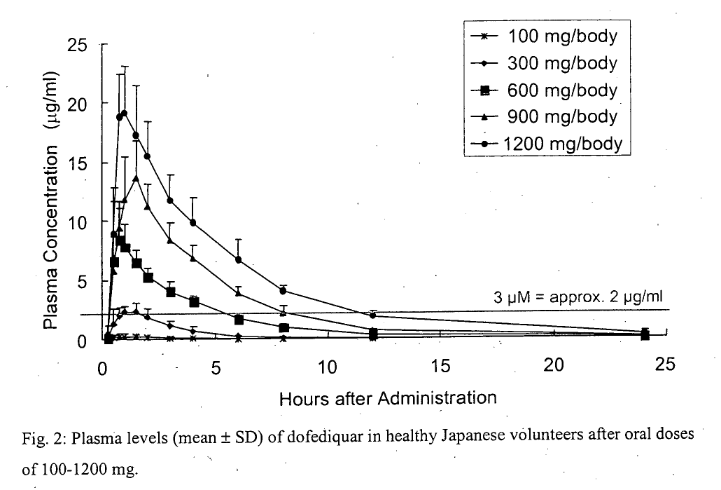

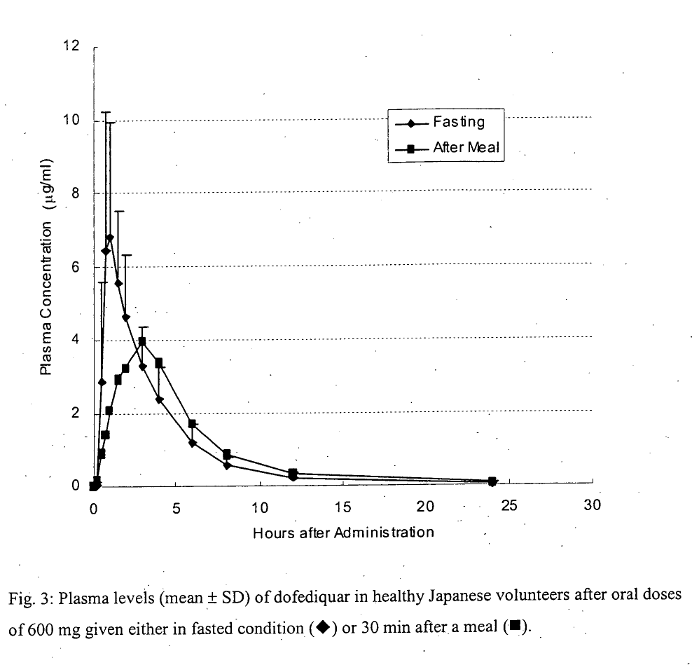

The pharmacokinetic parameters of dofediquar (MS-209) are investigated in fasted healthy male Japanese adults. The study design is described in Table 2. The plasma concentrations of dofediquar after single oral administration of increasing doses from 100 to 1200 mg to fasted healthy male Japanese adults is illustrated in FIGS. 2 and 3. The pharmacokinetic parameters are shown in Table 3. Dofediquar is not detected at a dose of 10 mg, but detected in one of 6 subjects at a dose of 30 mg. At doses of 100 mg or higher, plasma concentration of dofediquar reaches the peak at 0.75 to 1.5 hours after administration, and then declines in a monophasic manner. Cmax and AUC increase more steeply than expected from the dose increase, whereas Tmax does not change. Half-life also increases with increasing dose. The observed nonlinearity (i.e., overproportional increase of Cmax and AUC upon increasing dose) in the pharmacokinetic parameters is comparable to the results of the pharmacokinetic investigation in rats and dogs.

The effective concentration of dofediquar of about 3 μM (ca. 2 μg/mL) indicated in in vitro studies using drug-resistant cancer cell lines, is achieved at doses of 300 mg or higher.

The entire disclosures of all applications, patents and publications, cited herein and of corresponding U.S. Provisional Application Ser. No. 60/619,683, filed Oct. 19, 2004, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

TABLE 1
Effect of additional incubation with dofediquar (MS-209) on the growth of
K562/adriamycin-resistant cells after washing adriamycin (ADM) away
from culture medium

| | 2nd incu- | Growth rate (% of control) | | | | | |
|---|---|---|---|---|---|---|---|
| 1st incubation | bation | ADM 100 ng/ml | | | ADM 300 ng/ml | | |
| (1, 3, 6 hr) | (24 hr) | 1 hr | 3 hr | 6 hr | 1 hr | 3 hr | 6 hr |
| ADM | — | 93.4 | 102 | 92.0 | 88.1 | 104 | 100 |
| ADM + MS-209 | — | 95.0 | 74.7 | 11.7 | 79.5 | 28.3 | 35.5 |
| ADM + MS-209 | MS-209 | 81.0 | 51.7 | 18.8 | 43.6 | 14.9 | 14.4 |

TABLE 2
Outline of the study protocol

| | |
|---|---|
| Objectives | Evaluation of the safety and pharmacokinetics of MS-209 after single oral administration to |
| | healthy male adults. |
| Study Design | Placebo-controlled double-blind study for doses of 100 mg/body or more. Open-label study |
| | for doses of 10 mg and 30 mg/body. |
| Subject | Healthy male adults |
| Population | |
| Inclusion | 1) Those diagnosed as healthy in the prior examination |
| Criteria | 2) Those who do not take any drugs within 2 weeks before the study. |
| | 3) Those with body weight within 20% of the standard. |
| | 4) Those without history of hepatic, renal or heart disease. |
| | 5) Those aged 20-30 years. |
| Number of | Three subjects each receive 10 mg and 30 mg of the active drug. Six subjects each receive |
| Subjects | the active drug at doses of 100 mg or higher, while two subjects each received placebo for |
| | control. |
| Study Drug | 10 mg, 30 mg, 100 mg tablets of MS-209, and placebo tablet |
| Dosage | Fasted subjects receive MS-209 orally with 180 mL water. Dose is escalated sequentially |
| | from Level I to Level VI as follows: |
| | Level I (10 mg)→Level II (30 mg)→Level III (100 mg)→Level IV (300 mg)→Level V |
| | (600 mg)→Level VI (900 mg)→Level VII (1200 mg) |
| Observations & | Symptoms & signs, vital signs, ECG, hematological tests, serum chemistry tests, |
| Measurements | endocrinological tests, urinalysis, pharmacokinetics |

TABLE 3
Pharmacokinetic parameters of dofediquar in fasted healthy male volunteers.

| | | Pharmacokinetic parameters (mean ± S.D.) | | | | |
|---|---|---|---|---|---|---|
| MS-209 | Number of | Cmax | Tmax | Vd/F | t½ | AUC |
| Dose (mg) | Subjects | (μg/mL) | (h) | (L) | (h) | (μg · h/mL) |
| 10 | 3 | n.c. | n.c. | n.c. | n.c. | n.c. |
| 30 | 3 | n.c. | n.c. | n.c. | n.c. | n.c. |
| 100 | 6 | 0.47 ± 0.20 | 1.04 ± 0.40 | 190.7 ± 66.1 | 0.89 ± 0.37 | 0.72 ± 0.21 |
| 300 | 6 | 2.70 ± 0.74 | 1.04 ± 0.40 | 78.9 ± 25.3 | 1.17 ± 0.25 | 7.29 ± 2.54 |
| 600 | 6 | 10.11 ± 2.14 | 0.75 ± 0.22 | 65.2 ± 14.4 | 2.25 ± 0.47 | 32.47 ± 5.94 |
| 900 | 6 | 14.03 ± 2.48 | 1.67 ± 0.68 | 54.1 ± 12.3 | 2.39 ± 0.37 | 62.90 ± 9.67 |
| 1200 | 6 | 20.99 ± 2.15 | 1.00 ± 0.27 | 56.4 ± 10.4 | 3.10 ± 0.31 | 105.39 ± 20.10 |

n.c. not calculated

TABLE 2
Concentrations of dofediquar (MS-209) in plasma and tumor tissue
after single oral administration of 200 mg/kg in colon 26-bearing mice
(mean ± SD of N = 3).

Concentration of MS-209

| Time after administration (h) | Plasma (μg/ml) | Tumor (μg/g) |
|---|---|---|
| 0.5 | 25.6 ± 9.3 | 15.9 ± 8.1 |
| 1 | 33.0 ± 6.3 | 30.6 ± 7.5 |
| 2 | 27.3 ± 7.0 | 28.0 ± 9.4 |
| 5 | 24.6 ± 10 | 25.8 ± 10 |
| 24 | 0.61 ± 0.41 | 0.52 ± 0.49 |
| AUC(mg × h/mL or g tissue) | 368 | 376 |

What is claimed is:

1. A method of treating a patient with a disease that is subject to the development of multi-drug resistance (MDR) comprising administering to the patient one or more drugs active against the disease and one or more multi-drug resistance inhibitors wherein the plasma level-time profile of the MDR inhibitor(s) is matched to that of the active drug(s), or wherein the plasma concentration(s) of the MDR inhibitor(s) are above their thresholds of activity during the time periods that the plasma concentration(s) of the active drug(s) are above their activity threholds.

2. The method of claim 1 wherein the combination of active drug(s) and MDR inhibitor(s) is administered as first-line treatment.

3. The method of claim 2 wherein said administration prevents or ameliorates acquired MDR or reverses or ameliorates intrinsic MDR.

4. The method of claim 1 wherein in order to match the plasma level(s) of the active drug(s), the MDR inhibitor(s) is/are administered more often or less often than the active drug(s) during each treatment course, depending on the individual pharmacokinetic profiles of the active drug(s) and the MDR inhibitor(s).

5. The method of claim 2, wherein in order to match the plasma level(s) of the active drug(s), the MDR inhibitor(s) is/are administered more often or less often than the active drug(s) during each treatment course, depending on the individual pharmacokinetic profiles of the active drug(s) and the MDR inhibitor(s).

6. The method of claim 1 wherein the MDR inhibitor(s) is/are administered as slow-release formulation(s).

7. The method of claim 2, wherein the MDR inhibitor(s) is/are administered as slow-release formulation(s).

8. The method of claim 1 comprising administering doxorubicin and dofediquar.

9. The method of claim 2, comprising administrering doxorubicin and dofediquar.

10. The method of claim 8 comprising administering doxorubicin and dofediquar wherein per dose of doxorubicin, two doses of dofediquar are administered at an interval of 6-24 hours.

11. The method of claim 9 comprising administering doxorubicin and dofediquar wherein per dose of doxorubicin, two doses of dofediquar are administered at an interval of 6-24 hours.

12. The method of claim 1 comprising administering doxorubicin and dofediquar wherein per dose of doxorubicin, three doses of dofediquar are administered at an interval of 6-24 hours.

13. The method of claim 2, comprising administering doxorubicin and dofediquar wherein per dose of doxorubicin, three doses of dofediquar are administered at an interval of 6-24 hours.
